(19) United States
(12) Patent Application Publication
(10) Pub. No.: US 2008/0255547 A1
(43) Pub. Date: Oct. 16, 2008

(54) Uv Lamp for Treatment of the Skin

(75) Inventors: Giovanna Wagenaar-Cacciola, Eindhoven (NL); Arjan Van Der Pol, Roosendaal (NL); Matthias Born, Geldern (DE); Marc Janssen, Turnhout (BE); Marco Haverlag, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/065,623

(22) PCT Filed: Sep. 1, 2006
(86) PCT No.: PCT/IB06/53066

§ 371 Date: Mar. 4, 2008

(30) Foreign Application Priority Data
Sep. 6, 2005 (EP) 05108159.4

(51) Int. Cl. A61N 7/00; C09K 11/77

(52) U.S. Cl. 606/9; 313/487

(57) ABSTRACT

UV lamp for treatment of the skin, which lamp has a radiation capacity substantially entirely in the UV spectrum and in the red-light spectrum, wherein the radiation capacity of the lamp in the red light spectrum between 600 nm and 700 nm is at least 3% of the radiation capacity in the UV spectrum. In a first embodiment the lamp is a UV fluorescent lamp, having a first UV emitting phosphor and a second red light emitting phosphor, wherein the red light emitting phosphor is CeGdMgB₅O₁₀:Tb,Mn. In a second embodiment the lamp is a UV HID lamp, wherein the bulb of the lamp contains LiI and/or CaI₂ as a red light radiation salt.

The invention relates to a UV lamp for treatment of the skin, which lamp has a radiation capacity substantially entirely in the UV spectrum and in the red-light spectrum. Such a lamp is disclosed in U.S. Pat. No. 4,645,969.

The red-light component in the known lamp is produced by phosphors having colour wavelengths substantially below 600 nm, which improves the colour rendition, and avoids an unflattering appearance of the skin and clothes of the person using the lamp.

Irradiation with UV light is widely used for medical or aesthetical treatments like psoriasis and acne treatment, and for sun tanning. Fluorescent lamps specifically aimed at treatment of psoriasis are supplied by Philips™, and also various types of fluorescent and high-pressure sun tanning lamps are offered by several manufacturers. Moreover it is known that UV-radiation can be used to influence the concentration of NO molecules in the skin (see: Suschek C. V. et al., FASEB J 17, 2342-2344, 2003), which can influence the metabolic processes in the skin.

It is known that prolonged exposure to UV radiation can lead to photo-aging of the skin. Skin exposed to excessive UV radiation develops leathery texture, wrinkles, skin folds, sagging skin, warty growths called keratoses, freckling, and a yellow discoloration due to abnormal elastic tissue. People with the greatest risk of developing photo-aged skin are those with skins that burn easily, and who don't tan because they have less melanin in their skin. It is known that this is caused by the UV-induced breakdown of elastin in the skin, which reduces the elasticity of the skin. In photo-damaged skin the fiber elastic fibers are thick, highly curled and branched. Excessive sun exposure decreases the amount of collagen, because collagen is degraded due to the product of chronic inflammation released after UV damage (see: Berardesca E., Farinelli N., Rebbiosi G., Maibach H. I., Dermatologica 182, 1-6, 1991; Warren R., Gartstein V., Kligman A. M., Montagana W. et al., J. Am. Acad. Dermatol. 25, 751-60,1991).

The invention aims at a lamp of the above-mentioned type, which has more positive effects on the health of a person than prior art lamps.

According to the invention the radiation capacity of the lamp in the red light spectrum between 600 nm and 700 nm is at least 3% of the radiation energy capacity in the UV spectrum. Red light in this spectrum stimulates the formation of collagen in the skin and the proliferation of fibroblasts, which both improve the elasticity of the skin. The function of the red light is to promote the formation of ATP (proto-collagen) which is converted into collagen later (see: Weiss R. A., McDaniel D. H., Geronimus R. G., Sem. Cut. Med. Am. Surg., Vol. 22, No. 2, 2003, pp 93-106; Weiss R. A., McDaniel D. H., Geronemus R. G. et al., Lasers in surgery and medicine, 36-85, 91, 2005).

Preferably the radiation capacity of the lamp in the red light spectrum between 600 nm and 700 nm is at least 6% of the radiation energy capacity in the UV spectrum. Also preferably the radiation energy capacity of the lamp in the red light spectrum between 630 nm and 700 nm is at least 3%, preferably at least 6% of the radiation energy capacity in the UV spectrum.

In a first preferred embodiment the lamp is a UV fluorescent lamp, wherein the vessel of the lamp is coated with a first UV emitting phosphor and a second red light emitting phosphor, wherein the red light emitting phosphor is for instance CeGdMgB₅O₁₀:Tb,Mn (“CBTM phosphor”). This red light emitting phosphor has a radiation spectrum between 600 nm and 700 nm, with a peak at approximately 630-635 nm, and can for instance be used in the lamp described in U.S. Pat. No. 4,645,969, wherein it would replace the red-light emitting phosphors described therein. Although also MgGeO₆:Mn is known to have a radiation spectrum between 630 nm and 700 nm, it is unsuitable for the application of the current invention because it absorbs almost the entire UVA and UVB radiation, and therefore can not be applied in a quantity high enough to have the desired effect on the health of the skin. Effectively 6%-30%, preferably 10%-20%, (by weight) of the phosphor mix consists of the red light emitting phosphor, whereby the above described radiation energy levels can be achieved.

In a second preferred embodiment the lamp is a UV HID lamp, wherein the vessel of the lamp contains a first UV radiating salt and a second red light radiating salt, and wherein the red light radiation salt is LiI and/or Cal₂. The red light radiation salt can for instance be added to a lamp of the type Philips™ Cleo UV HPA 400S, which is a high pressure iron/cobalt metal halide lamp without any phosphor coating, having a nominal power of 400 W, mainly radiating in the UVA spectrum. In a lamp having a nominal power between 100 W and 800 W the vessel preferably contains between 0.05 and 1 mg, more preferably between 0.10 mg and 0.5 mg, still more preferably between 0.2 mg and 0.3 mg of the red light radiation salt.

The preferred dose of red light between 630 nm and 700 nm during one sun tanning session wherein the lamp of the invention is used, is at least 2 J per cm² skin, compared to a preferred dose of UV light of approximately 20 J per cm² skin.

The invention will be further explained by means of exemplary embodiments and with reference to the drawings, wherein:

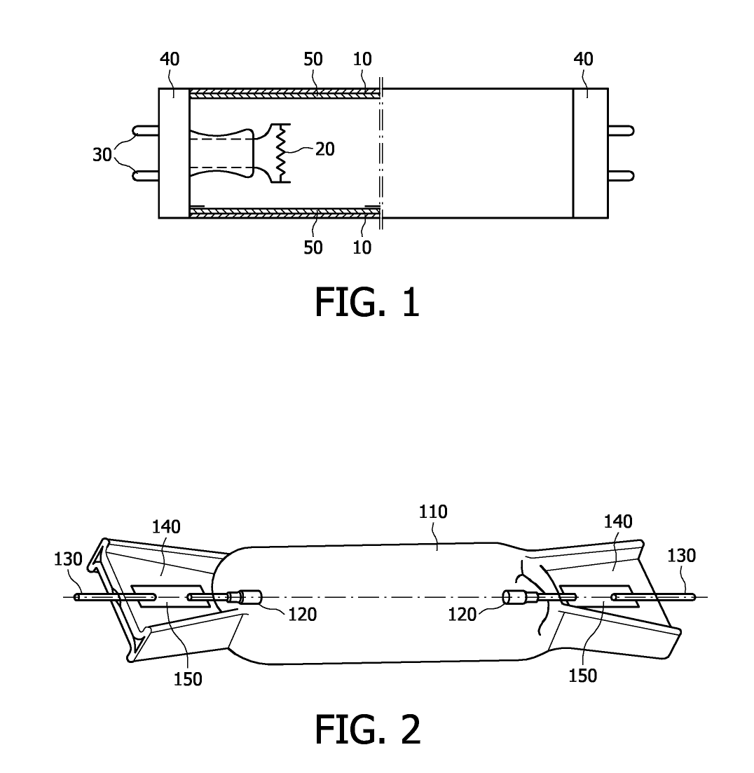

FIG. 1 shows a low-pressure mercury discharge UV lamp (partly in section view); and

FIG. 2 shows a high-pressure metal halide discharge UV lamp.

According to FIG. 1 a low pressure mercury discharge lamp for skin tanning purposes comprises a tubular soda lime silicate glass vessel 10, having electrodes 20 at both ends. The electrodes are electrically connected with two connectors 30, which extend from end caps 40.

The vessel 10 is filled with a well-known low-pressure inert gas filling, such as argon, and a small amount of mercury. Since this kind of low pressure mercury discharge lamp is well-known in the art, apart from the applied phosphors, further details of the lamp are not explained here.

The inner surface of the vessel 10 is coated with a phosphor mixture 50 of at least two phosphors. The first phosphor is a phosphor that radiates in the UV spectrum, such as SrB₄O₇:Eu (“SBE phosphor”) or BaSi₂O₅:Pb (“BSP phosphor”). The second phosphor is CeGdMgB₅O₁₀:Tb,Mn (“CBTM phosphor”), a phosphor that radiates red light in the spectrum between 600 nm and 700 nm when the lamp is ignited. Approximately 15% (by weight) of the phosphor mix consists of the CBTM phosphor.

According to FIG. 2 a high pressure metal halide discharge lamp for skin tanning purposes comprises a tubular quartz vessel 110, having electrodes 120 at both ends. The electrodes 120 are electrically connected with two connectors 130 by means of Mo-foils 150, which extend through a pinch 140 at both ends.

The vessel 110 is filled with a well-known high-pressure inert gas filling, such as argon and a small amount of mercury, as well as a cobalt and iron metal halide salt mix. Since this kind of high-pressure metal halide discharge lamp is well-known in the art (it is known as the Philips™ Cleo Suntanning Lamp HPA 400S), apart from the additional salts, further details of the lamp are not explained here.

The vessel 110 further contains approximately 0.25 mg LiI and/or CaI₂ as a red light radiation salt. When the lamp is ignited these salts radiate red light in the spectrum between 600 nm and 700 nm.

1. UV lamp for treatment of the skin, which lamp has a radiation energy capacity substantially entirely in the UV spectrum and in the red-light spectrum, characterized in that the radiation energy capacity of the lamp in the red light spectrum between 600 nm and 700 nm is at least 3% of the radiation energy capacity in the UV spectrum.

2. UV lamp according to claim 1, wherein the radiation energy capacity of the lamp in the red light spectrum between 600 nm and 700 nm is at least 6% of the radiation energy capacity in the UV spectrum.

3. UV lamp according to claim 1, wherein the radiation energy capacity of the lamp in the red light spectrum between 630 nm and 700 nm is at least 3%, preferably at least 6% of the radiation energy capacity in the UV spectrum.

4. UV lamp according to claim 1, wherein the lamp is a UV fluorescent lamp, wherein the transparent vessel of the lamp is coated with a first UV emitting phosphor and a second red light emitting phosphor.

5. UV lamp according to claim 4, wherein the red light emitting phosphor is CeGdMgB₅O₁₀:Tb,Mn.

6. UV lamp according to claim 4, wherein 6%-30%, preferably 10%-20%, (by weight) of the phosphor mix consists of the red light emitting phosphor.

7. UV lamp according to claim 1, wherein the lamp is a UV HID lamp, wherein the vessel of the lamp contains a first UV radiating salt and a second red light radiating salt, and wherein the red light radiating salt is LiI and/or CaI₂.
